(19) United States
(12) Patent Application Publication
(10) Pub. No.: US 2024/0066254 A1
(43) Pub. Date: Feb. 29, 2024

(54) Tube with an integrated cleanout port

(71) Applicant: Matthew J. Marini, Gold River, CA (US)

(72) Inventor: Matthew J. Marini, Gold River, CA (US)

(21) Appl. No.: 17/900,691

(22) Filed: Aug. 31, 2022

(51) Int. Cl. A61M 16/08; F16L 11/15; F16L 41/02; F16L 58/00

(52) U.S. Cl. CPC A61M 16/0808; F16L 11/15; F16L 41/021; F16L 58/00

(57) ABSTRACT

A tube with an integrated cleanout port includes a medical-grade tube, a suction adaptor, and a check valve, The medical-grade tube that connect between the patient's endotracheal tube and the Y-pipe of the mechanical ventilator includes a tubular body, a first connector end, and a second connector end. The suction adaptor is laterally positioned to the tubular body and hermetically connected to the tubular body that may be corrugated or not corrugated. The check valve is hermetically connected within the suction adaptor, wherein the tubular body is in fluid communication with the suction adaptor through the check valve. Due to the configuration of the check valve, any fluid buildup such as condensation, sputum, blood, and mucous within the tubular body can easily be removed via the suction adaptor without having to shut off the mechanical ventilator or break the circuit which could potentially cause harm to the patient.

### FIELD OF THE INVENTION

The present invention relates generally to a tubing of the mechanical ventilators. More specifically, the present invention is attached in between the patient's endotracheal tube and the Y-pipe of the mechanical ventilator to clean up fluid buildup.

### BACKGROUND OF THE INVENTION

Mechanical ventilation, assisted ventilation or intermittent mandatory ventilation (IMV), is the medical term for using a machine called a ventilator to provide artificial ventilation fully or partially. Mechanical ventilation helps move air into and out of the lungs, with the main goal of helping the delivery of oxygen and removal of carbon dioxide. Mechanical ventilation is used for many reasons, including to protect the airway due to mechanical or neurologic cause, to ensure adequate oxygenation, or to remove excess carbon dioxide from the lungs. Various healthcare providers are involved with the use of mechanical ventilation and people who require ventilators are typically monitored in an intensive care unit. The main risk of mechanical ventilation is an infection, as the artificial airway (breathing tube) may allow germs to enter the lung. Any kind of fluid buildup, such as condensation, sputum, blood, and mucous, that generally occurs within the breathing tube can significantly increase the aforementioned infection thus jeopardizing the patient's health.

An objective of the present invention is to provide a tube with an integrated cleanout port so that the caregivers can clean any fluid buildup within the breathing tube. More specifically, an integrated check valve of the present invention allows the caregivers to full remove any buildup within the breathing tube. As a result, the patient is able to breath in clean a flow of oxygen and also maintain pressure within the circuit which may be needed to support the patients breathing while being connected to the mechanical ventilator.

### BRIEF DESCRIPTION OF THE DRAWINGS

FIG. 1 is a top perspective view of the present invention without showing the cap.

FIG. 2 is a top view of the present invention without showing the cap.

FIG. 3 is a top view of the present invention, showing the cap.

FIG. 4 is a side view of the present invention showing the plane upon which a cross sectional view is taken shown in FIG. 5.

FIG. 5 is a cross section view of the present invention taken along line A-A of FIG. 4, showing the internal connection of the check valve within the suction adaptor.

### DETAIL DESCRIPTIONS OF THE INVENTION

All illustrations of the drawings are for the purpose of describing selected versions of the present invention and are not intended to limit the scope of the present invention.

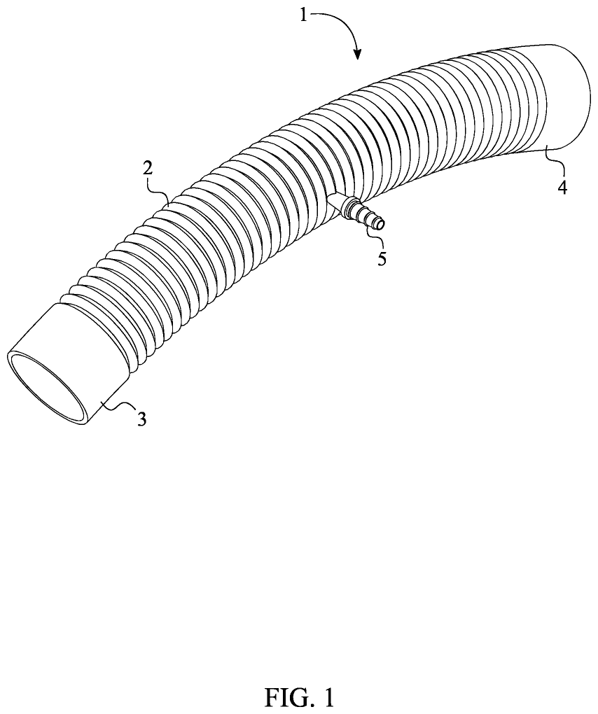

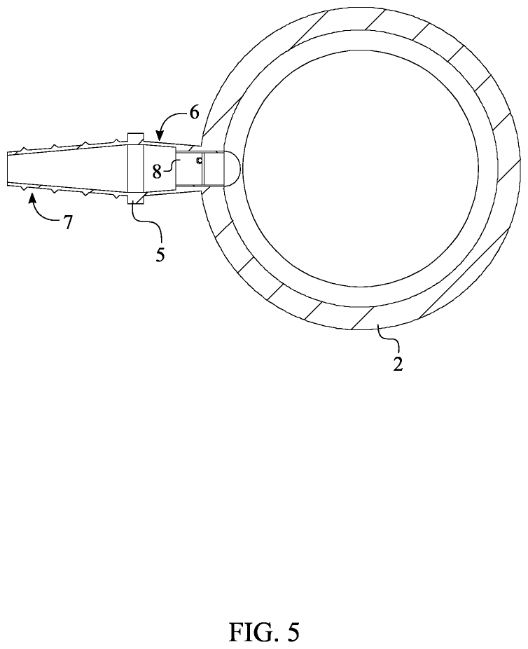

The present invention is a tube with an integrated cleanout port so that the caregivers can clean any fluid buildup within the breathing tube. Due to the operation of the mechanical ventilator and the medical conditions of the patient, fluid buildup (condensation, sputum, blood, and mucous) generally occurs in between the patient's endotracheal tube and the Y-pipe of the mechanical ventilator. The present invention is mounted in between the patient's endotracheal tube and the Y-pipe of the mechanical ventilator thus allowing the caregiver to easily remove the aforementioned fluid buildup without having the shut down the mechanical ventilator. The present invention comprises a medical-grade tube 1, a suction adaptor 5, and a check valve 8 as shown in FIG. 1 and FIG. 5.

In reference to the general configuration of the present invention, the medical-grade tube 1 that mounts in between the patient's endotracheal tube and the Y-pipe of the mechanical ventilator comprises a tubular body 2, a first connector end 3, and a second connector end 4 as shown in FIGS. 1-2 and FIG. 5. The suction adaptor 5 is laterally positioned to the tubular body 2 and hermetically connected to the tubular body 2 so that the caregiver can remove fluid buildup from the tubular body 2. The check valve 8 is hermetically connected within the suction adaptor 5 thus allowing the tubular body 2 to be in fluid communication with the suction adaptor 5 through the check valve 8. In other words, the check valve 8 functions as a one-way valve to only remove fluid buildup and does not allow any outside elements to enter into the tubular body 2.

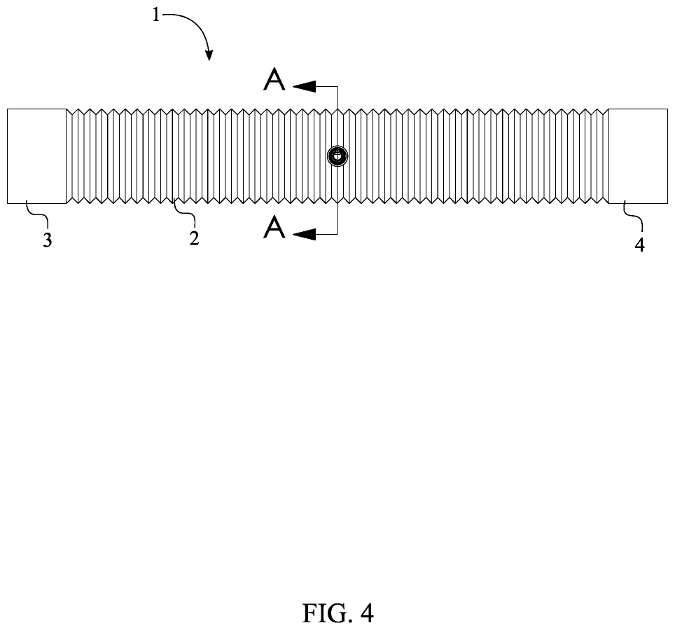

The medical-grade tube 1 functions as the main interconnecting tubular component in between the patient's endotracheal tube and the Y-pipe of the mechanical ventilator. In reference to FIG. 1-4, the first connector end 3 and the second connector end 4 are oppositely positioned of each other about the tubular body 2. The first connector end 3 is hermetically connected to the tubular body 2. The second connector end 4 is hermetically connected to the tubular body 2, opposite of the first connector end 3. More specifically, the first connector end 3 is in fluid communication with the second connector end 4 through the tubular body 2 so that the delivery of oxygen and removal of carbon dioxide can be carried out. Furthermore, the first connector end 3 is configured to hermetically attach with the patient's endotracheal tube. The second connector end 4 is configured to hermetically attach with the Y-pipe of the mechanical ventilator. As a result, the caregiver can easily set up the operation of the mechanical ventilator without any modifications to the endotracheal tube and the Y-pipe of the mechanical ventilator.

In reference to FIG. 1-3, the tubular body 2 is preferably a corrugated tubular body 2 so that the overall length of the medical-grade tube 1 can be adjusted in between the first connector end 3 and the second connector end 4. However, the tubular body 2 can also have circular cross-section or any other type of geometric cross-section as the functionality of the medical-grade tube 1 is not limited by the cross-sectional shape of the tubular body 2. A diameter of the tubular body 2 is less than 4 inches to match the diameter of the existing tubing associated with the mechanical ventilator.

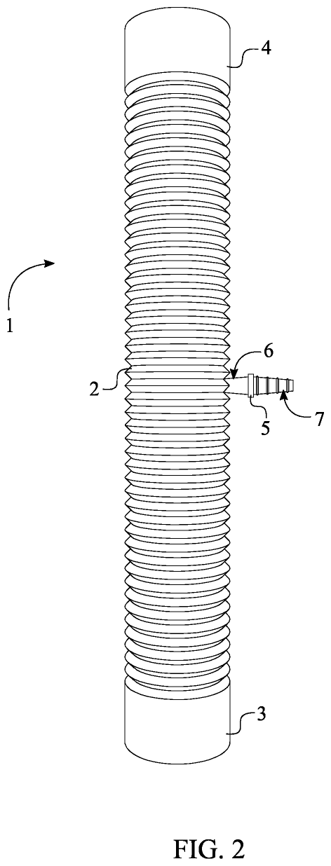

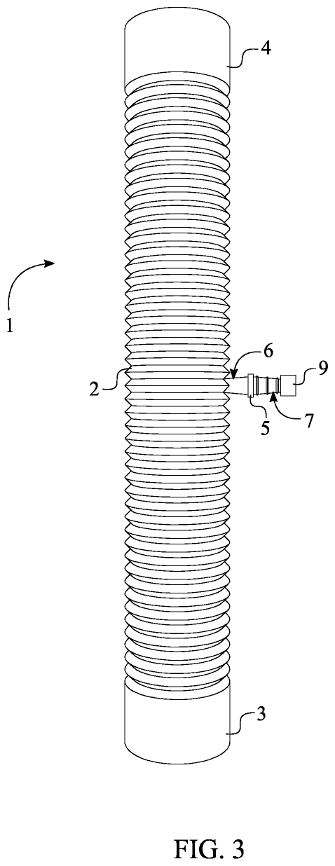

In reference to FIG. 2-3, the suction adaptor 5 comprises a proximal port-end 6 and a distal port-end 7. More specifically, the proximal port-end 6 is in fluid communication with the distal port-end 7 so that the suction adaptor 5 can provide a continuous channel from one end to the opposite end. The proximal port-end 6 is hermetically connected to the tubular body 2 as the check valve 8 is hermetically connected within the proximal port-end 6. In other words, when fluid buildup is removed from the tubular body 2, the flow of fluid is discharged into the suction adaptor 5 through the check valve 8. The distal port-end 7 is offset from the tubular body 2 and configured to receive a handheld suction device or a wall suction apparatus of the hospital room. In other words, the suction power necessary to remove any fluid buildup is supplied through the distal port-end 7.

In reference to FIG. 3, the present invention further comprises a cap 9. More specifically, the cap 9 is hermetically mounted to the distal port-end 7 of the suction adaptor 5. The attachment between the cap 9 and the distal port-end 7 can be a threaded attachment, a friction-fit attachment, or any other attachment mechanism that seals the suction adaptor 5. As a result, the cap 9 prevents any outside elements such as dust and debris from entering into the suction adaptor 5 via the distal port-end 7. Furthermore, the cap 9 also functions as a secondary barrier for the tubular body 2 so that any outside elements do not enter into the medical-grade tube 1 via the failed check valve 8.

Although the invention has been explained in relation to its preferred embodiment, it is to be understood that many other possible modifications and variations can be made without departing from the spirit and scope of the invention as hereinafter claimed.

What is claimed is:

1. A tube with an integrated cleanout port comprising:
a medical-grade tube;
a suction adaptor;
a check valve;
the medical-grade tube comprising a tubular body, a first connector end, and a second connector end;
the suction adaptor being laterally positioned to the tubular body;
the suction adaptor being hermetically connected to the tubular body;
the check valve being hermetically connected within the suction adaptor; and
the tubular body being in fluid communication with the suction adaptor through the check valve.

2. The tube with an integrated cleanout port as claimed in claim 1 comprising:
the first connector end and the second connector end being oppositely positioned of each other about the tubular body;
the first connector end being hermetically connected to the tubular body;
the second connector end being hermetically connected to the tubular body, opposite of the first connector end; and
the first connector end being in fluid communication with the second connector end through the tubular body.

3. The tube with an integrated cleanout port as claimed in claim 1, wherein the tubular body is a corrugated tubular body.

4. The tube with an integrated cleanout port as claimed in claim 1, wherein a diameter of the tubular body is less than 4 inches.

5. The tube with an integrated cleanout port as claimed in claim 1 comprising:
the suction adaptor comprising a proximal port-end and a distal port-end;
the proximal port-end being in fluid communication with the distal port-end;
the proximal port-end being hermetically connected to the tubular body;
the check valve being hermetically connected within the proximal port-end; and
the distal port-end being offset from the tubular body.

6. The tube with an integrated cleanout port as claimed in claim 1 comprising:
a cap; and
the cap being hermetically mounted to a distal port-end of the suction adaptor.

7. A tube with an integrated cleanout port comprising:
a medical-grade tube;
a suction adaptor;
a check valve;
the medical-grade tube comprising a tubular body, a first connector end, and a second connector end;
the suction adaptor comprising a proximal port-end and a distal port-end;
the proximal port-end being in fluid communication with the distal port-end;
the suction adaptor being laterally positioned to the tubular body;
the suction adaptor being hermetically connected to the tubular body;
the proximal port-end being hermetically connected to the tubular body;
the check valve being hermetically connected within the suction adaptor;
the check valve being hermetically connected within the proximal port-end;
the distal port-end being offset from the tubular body; and
the tubular body being in fluid communication with the suction adaptor through the check valve.

8. The tube with an integrated cleanout port as claimed in claim 7 comprising:
the first connector end and the second connector end being oppositely positioned of each other about the tubular body;
the first connector end being hermetically connected to the tubular body;
the second connector end being hermetically connected to the tubular body, opposite of the first connector end; and
the first connector end being in fluid communication with the second connector end through the tubular body.

9. The tube with an integrated cleanout port as claimed in claim 7, wherein the tubular body is a corrugated tubular body.

10. The tube with an integrated cleanout port as claimed in claim 7, wherein a diameter of the tubular body is less than 4 inches.

11. The tube with an integrated cleanout port as claimed in claim 7 comprising:
a cap; and
the cap being hermetically mounted to the distal port-end of the suction adaptor.

12. A tube with an integrated cleanout port comprising:
a medical-grade tube;
a suction adaptor;
a check valve;
a cap;
the medical-grade tube comprising a tubular body, a first connector end, and a second connector end;
the suction adaptor comprising a proximal port-end and a distal port-end;
the proximal port-end being in fluid communication with the distal port-end;
the suction adaptor being laterally positioned to the tubular body;
the suction adaptor being hermetically connected to the tubular body;
the proximal port-end being hermetically connected to the tubular body;
the check valve being hermetically connected within the suction adaptor;
the check valve being hermetically connected within the proximal port-end;
the distal port-end being offset from the tubular body;
the cap being hermetically mounted to the distal port-end of the suction adaptor; and
the tubular body being in fluid communication with the suction adaptor through the check valve.

13. The tube with an integrated cleanout port as claimed in claim 12 comprising:
the first connector end and the second connector end being oppositely positioned of each other about the tubular body;
the first connector end being hermetically connected to the tubular body;
the second connector end being hermetically connected to the tubular body, opposite of the first connector end; and
the first connector end being in fluid communication with the second connector end through the tubular body.

14. The tube with an integrated cleanout port as claimed in claim 12, wherein the tubular body is a corrugated tubular body.

15. The tube with an integrated cleanout port as claimed in claim 12, wherein a diameter of the tubular body is less than 4 inches.
